(19) United States
(12) Patent Application Publication
(10) Pub. No.: US 2005/0245905 A1
(43) Pub. Date: Nov. 3, 2005

(54) Local drug-delivery system

(76) Inventors: Steven Paul Schmidt, Akron, OH (US); Stephanie Lopina, North Canton, OH (US); Deenu George Kanjickal, Akron, OH (US); Mary Michelle Evancho-Chapman, Kent, OH (US)

Correspondence Address: ROETZEL AND ANDRESS, 222 SOUTH MAIN STREET, AKRON, OH, 44308, US

(21) Appl. No.: 10/836,787

(22) Filed: Apr. 30, 2004

(51) Int. Cl. A61K 9/22

(52) U.S. Cl. 604/891.1; 424/425

(57) ABSTRACT

A medical device for delivering a pharmacological agent to a target region of tissue. The device includes a resilient carrier material shaped to substantially encircle the target region and a first pharmacological agent provided to the carrier material. The first pharmacological agent is to be released from the carrier material during a first period of time and introduced to the target region.

### FIELD OF THE INVENTION

The invention relates generally to drug-delivery systems, and more particularly, to a method and device for controllably delivering a pharmacological agent to a local target region of tissue over a desired period of time.

### BACKGROUND OF THE INVENTION

The immune system of the human body can overcompensate for the presence of foreign matter within the body, even when the foreign matter has been introduced in an effort to rectify a potentially fatal condition. For example, hemodialysis is a conventional method for treating a vast majority of End-Stage Renal Disease (ESRD) patients. Hemodialysis access grafts can experience failure rates of up to 50-80% at one year due in part to venous stenosis. Venus stenosis is caused by intimal hyperplasia (1H) arising from graft compliance mismatch, flow turbulence, shear stress, vessel stretch, surgical trauma, mural ischemia, and luminal accumulation of various biochemical factors released from deposited fibrin and platelets. IH is a normal response to tissue injury and involves both the migration of predominantly smooth muscle cells out of the tunica media into the tunica intima of the vessel wall and their subsequent proliferation. Extracellular matrix is later synthesized and deposited by these rapidly growing cells, resulting in a hardened layer of mural thickening comprised of smooth-muscle cells, fibroblasts, various blood cells, and structural proteins. The thickened portion of the vascular wall extends into the interior of the blood vessel, decreasing the interior diameter of the blood vessel and causing a reduction of the blood flow therein. Such a combination can induce thrombosis, and ultimately failure of the hemodialysis graft.

Recent studies have indicated that drugs such as Cyclosporin A (CyA) were able to inhibit the development of IH in a canine test subject. However, because CyA is an immuno-suppressive agent, it is not recommended for systematic administration to ESDR patients, and instead, should be delivered locally to the desired target-tissue region. Conventional methods for local delivery of a therapeutic substance such as CyA typically require the particular substance to be injected into the target region. But this is also problematic in that the entire dose of the therapeutic substance is simultaneously delivered to the target region.

To prolong the period during which locally administered therapeutic substances are effective, attempts have been made to encapsulate the medication in a degradable capsule that can be injected into or applied onto affected tissue regions. The rate at which the encapsulated drug is delivered to the affected tissue regions can be controlled by selecting suitable materials, capsule dimensions, and other such factors. The encapsulated medications can be injected into the bloodstream or coated onto the affected tissue regions before the surgical procedure is completed with the closing of the surgical incision, allowing the medication to gradually escape from the capsule as the capsule degrades. Capsules of medication administered in this manner can become dislodged from tissue on which they have been coated, and are distributed throughout the body when injected into the bloodstream.

More recently, hydrogels have been used as carriers to enable the local delivery of therapeutic substances to affected tissue regions. Capsules containing a therapeutic substance are suspended in the hydrogel to be applied as a paste to the areas of interest. As the capsules degrade, the escaping therapeutic substance diffuses through the hydrogel. The therapeutic substance eventually reaches the surfaces of the hydrogel and is delivered to the adjacent environments as well as the targeted-tissue region. Since the therapeutic substance is also delivered to the other environments exposed to the surfaces of the hydrogel, it is difficult to accurately predict the dosage of the therapeutic substance that is actually delivered to the target region.

A hydrogel processed into a flexible sheet has been proposed for the local delivery of a therapeutic substance to a target tissue region. Various compositions were analyzed for the formation of a hydrogel that afforded the flexibility needed to be able to wrap the hydrogel sheet provided with the therapeutic substance around the target tissue region during a surgical procedure. Although this hydrogel sheet was adaptable to accommodate a target tissue region of any shape, it proved to be difficult to apply and secure to the target region of tissue.

Accordingly, there is a need in the art for a drug-delivery device and method for delivering one or more pharmacological agents to a local target area of tissue. The device and method should be adaptable to deliver a pharmacological agent to shaped target regions of tissue, should readily accommodate installation to a variety of target regions, and should be able to deliver more than one pharmacological agent to the target region, possibly during multiple different time periods.

### SUMMARY OF THE INVENTION

It is an objective of the invention to controllably delivery a pharmacological agent to a target region of tissue. It is another objective of the present invention to controllably deliver more than one pharamacological agent to the target region at a desired rate.

In accordance with one aspect, the present invention provides a medical device for delivering a pharmacological agent to a target region of tissue. The device includes a resilient carrier material shaped to substantially encircle the target region; and a first pharmacological agent provided to the carrier material, wherein the first pharmacological agent is to be released from the carrier material during a first period of time and introduced to the target region.

In accordance with one aspect, the present invention provides a device for delivering a pharmacological agent to a target region of tissue. The device includes a pliant-material matrix to be positioned adjacent to the target region; a first pharmacological agent generally homogeneously incorporated into the pliant-material matrix; and a second pharmacological agent provided to the pliant-material matrix, wherein the first pharmacological agent is released from the pliant-material matrix and introduced at the target region during a first period of time beginning substantially immediately after the pliant material is positioned proximate to the target region. The second pharmacological agent is released from the pliant-material matrix and introduced at the target region during a second period of time that extends beyond the expiration of the first period of time.

In accordance with another aspect, the present invention also provides a device for delivering a pharmacological agent to a target region of tissue. The device includes a pliant material having a first surface to be placed in contact with the target region and a second surface that is not in contact with the target region when the first surface is in contact with the target region; a first pharmacological agent provided to the pliant material, wherein the first pharmacological agent is to be released from the pliant material and introduced to the target region at an interface between the first surface and the target region; and a barrier layer provided positioned to minimize release of the first pharmacological agent from the pliant material to an ambient environment at the second surface.

In accordance with yet another aspect, the present invention also provides a device for delivering a first pharmacological agent and a second pharmacological agent to a target region of tissue. The device includes a carrier material having a first terminal-end portion positioned adjacent to a second terminal-end portion and defining a generally-cylindrical interior passage and a first pharmacological agent provided to the carrier material, wherein the first pharmacological agent is to be released from the carrier material during a first period of time and introduced to the target region. The device further includes a second pharmacological agent provided to the carrier material, wherein the second pharmacological agent is released from the carrier material and introduced at the target region during a second period of time that extends beyond the expiration of the first period of time. A barrier layer is provided to a surface of the carrier material to minimize release of at least one of the first pharmacological agent the second pharmacological agent from the carrier material to an ambient environment at an undesirable location.

### BRIEF DESCRIPTION OF THE DRAWINGS

The foregoing and other features and advantages of the present invention will become apparent to those skilled in the art to which the present invention relates upon reading the following description with reference to the accompanying drawings, in which:

FIG. 1 illustrates an example of a drug-delivery device in accordance with an embodiment of the present invention; and

FIG. 2 illustrates an application of the device shown in FIG. 1 as used adjacent to a target regions of tissue forming parts of an artery, vein and a dialysis access graft.

### DETAILED DESCRIPTION OF AN ILLUSTRATIVE EMBODIMENT

Certain terminology is used herein for convenience only and is not to be taken as a limitation on the present invention. Further, in the drawings, certain features may be shown in somewhat schematic form.

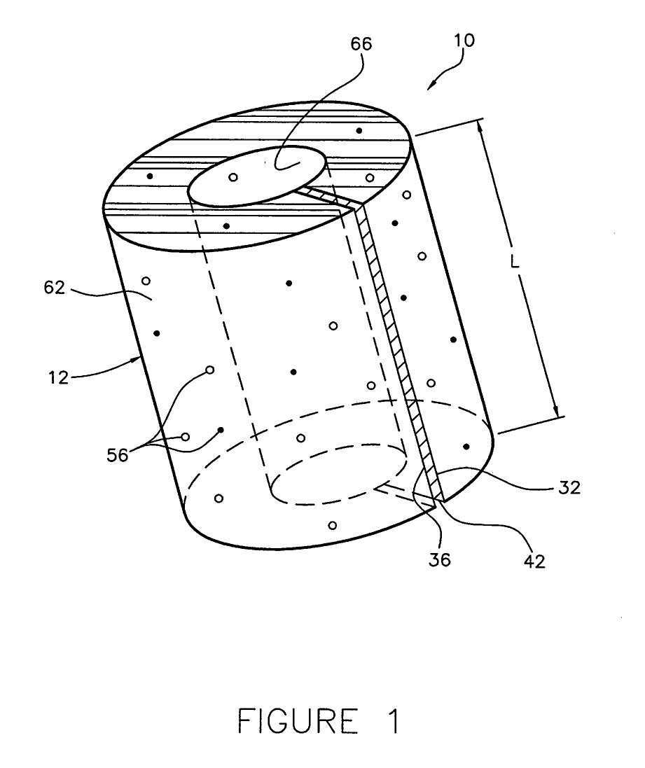

FIG. 1 shows an embodiment of a medical device 10 according to the present invention for delivering a pharmacological agent to a target region of tissue (not shown). The device comprises a resilient carrier material 12 shaped to substantially encircle the target region, and a first pharmacological agent provided to the carrier material. The first pharmacological agent is to be released from the carrier material 12 during a first period of time and introduced to the target region.

The carrier material 12 of the device 10 shown in FIG. 1 is formed from a three-dimensional matrix, such as a hydrogel matrix, that can be implanted in a patient during a surgical procedure and provides the device 10 with a resilient shape. In this manner, the device 10 can be installed by applying a force causing elastic deformation of the carrier material 12, placing the carrier material 12 in close proximity to a target tissue region, and then releasing the force applied to the carrier material 12. Once the force applied to the carrier material 12 causing elastic deformation is released, the resilient nature of the hydrogel matrix will exert a force that tends to return the carrier material 12 approximately to its original, unbiased shape. As shown in FIG. 1, the original, unbiased shape of the carrier material 12 is cylindrical, defining a generally-cylindrical interior passage extending longitudinally through the carrier material 12. The cylindrical embodiment of the carrier material 12 includes a first terminal end 32 proximate to a second terminal end 36 of the carrier material 12. The first and second terminal ends 32, 36 can either be coupled together, or placed adjacent to each other forming a longitudinal aperture 42 that extends along the axial length L of the cylindrical carrier material 12. Carrier materials having shapes other than cylindrical, such as oblong, oval, and the like are also contemplated within the scope of the present invention.

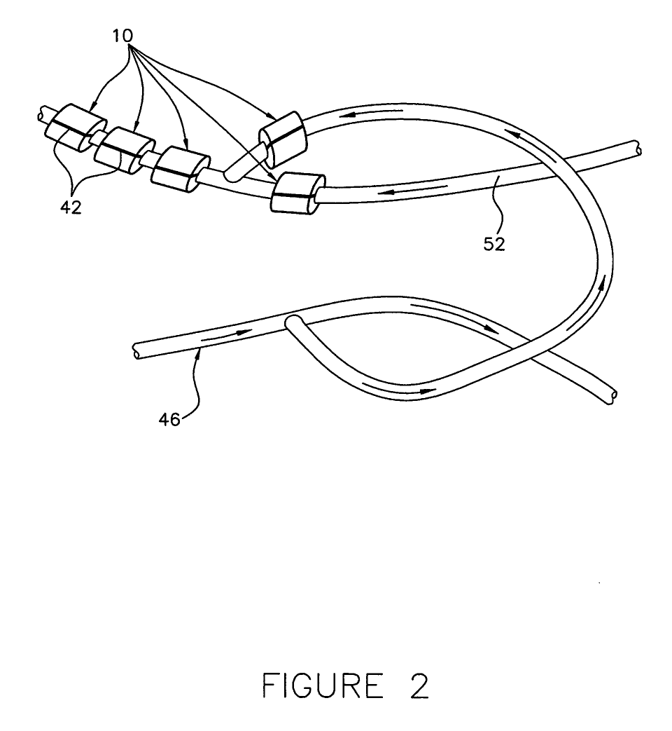

Preferably, when the device 10 is installed on a vascular channel such as an artery 46, vein 52 (FIG. 2), and any other object within the patient's body, the carrier material 12 will substantially encircle the target region of tissue to which the pharmacological agent is to be delivered. The resilient force exerted by the hydrogel matrix will cause the carrier material 12 to grasp, or clamp onto the object. Although the device 10 is described below for use on a target region of tissue, it is understood that the target region of tissue, in addition to arteries and veins, encompasses other suitably-shaped structures to which the device 10 of the present invention could be coupled for delivering a pharmacological agent. Examples include portions of the cardiovascular system such as the aorta, vena cava, large branches of the lymphatic drainage, and the like; portions of the pulmonary system such as the trachea, bronchi, bronchial branches, and the like; portions of the reproductive system such as fallopian tubes, cervix, vas deferens, epididymis, and the like; portions of the urinary tract such as the ureter, urethra, and the like; portions of the digestive system such as the esophagus, duodenum, small intestine, large intestine, bile duct, hepatic duct, pancreatic duct, and the like; and orthopedic structures such as tendons, ligaments, long bones, and the like. While this listing of potential applications is helpful for understanding some potential uses of the present invention, this list is not exhaustive.

The hydrogel matrix of the present invention forms a gelatinous three-dimensional carrier material 12 capable of reversible deformation as discussed above. By resilient, it is meant that the shape of the carrier material 12 can be elastically deformed to facilitate installation of the device 10 adjacent to a target regions of tissue, while exerting a force biasing the carrier material 12 toward its original shape once the device 10 is in place. It is not required for the carrier material 12 to return to its exact original shape, however, the resilient carrier material exerts a force that at least partially returns the carrier material 12 to a shape resembling the original shape. This provides a securing force that minimizes the tendency of the device 10 to move from the target region.

In one embodiment, hydrogel, as used herein, is a substance formed when an organic polymer (natural or synthetic) is cross-linked via covalent, ionic, or hydrogen bonds to create a three-dimensional open-lattice structure which entraps water molecules to form a gel. In another embodiment, hydrogels are water-swollen networks of hydrophilic homopolymers or copolymers. The hydrogel matrix can also entrap a solution including a pharmacological agent. Naturally occurring and synthetic hydrogel forming polymers, polymer mixtures and copolymers may be utilized as hydrogel precursors. Suitable hydrogel matrices comprise an aqueous component and a polymeric component dispersed throughout the aqueous component. Suitable organic polymers for the formation of a hydrogel include, but are not limited to, a polyurethane, polycarboxylic acid, polyorthoester, aliphatic polyester, polyanhydride, polysaccharide, polyamide, polyether, polyvinyl alcohol, polyethylene oxide, polyethylene glycol, protein, polypeptide, silicone based polymer, polyacrylamide, photopolymerizable monomer, polyglycolic acid, polylactic acid, poly(lactic-co-glycolic) acid, polycaprolactone, modified starch, gelatin, cellulose and its derivates, polyacrylic acid, polymethacrylic acid, polyhydroxybutyrate, polydioxanon, poly(ethylene vinyl acetate), polyethylene terephthalate, poly(vinylpyrrolidone), polytetrafluoroethylene, polyolefin, epoxide, poly(2-hydroxyethylmethacrylate) polyphosphazene polymer, fluoropolymer, polyamino acid, polyimine, polyphosphate, polysiloxane, polyvinyl ether, polyhydroxy acid, polyalkyl carbonate, albumin, fibrin, chitosan, alginate, poly(methylmethacrylate), collagen, polyphosphoester, hyaluronic acid, phospholipid, cyanoacrylate, polypropylene oxide, and any combination thereof.

The hydrogel matrix can be shaped into generally any desired shape. The hydrogel matrix can be prepared in the desired shape such as by molding and other preparatory techniques. Similarly, the desired shape of the hydrogel matrix to form the carrier material 12 can be attained by performing a post-preparation operation on an existing hydrogel matrix such as by cutting a hydrogel matrix with a heated element, boring tool, and the like into the desired shape.

Due to the properties of the hydrogel matrix, the carrier material 12 can absorb a pharmacological agent that is to be released from the carrier material 12 and introduced to the target region of tissue. According to one embodiment, a pharmacological agent is absorbed by the hydrogel matrix of the carrier material 12, thereby becoming generally homogeneously incorporated in the carrier material 12. Alternately, a pharmacological agent that is insoluble in the hydrogel matrix of the carrier material 12 can be selected to form a suspension or dispersion within the carrier material 12 as described in detail below. According to another embodiment, a first pharmacological agent can be generally homogeneously incorporated throughout the carrier material 12, and a second pharmacological agent can be suspended within the carrier material 12. If a first pharmacological agent is homogeneously incorporated within a carrier material, then this description is directed to characterizing both the concentration of a pharmacological agent throughout a carrier material as well as describing the phase in which the component is dispersed. Specifically, a homogeneously-dispersed pharmacological agent is dispersed substantially evenly throughout a liquid phase of a hydrogel matrix. In one embodiment, the pharmacological agent is homogeneously incorporated in a carrier material when it is evenly dispersed in at least a liquid phase that has been absorbed by the carrier material. In contrast, depicting a pharmacological agent as being suspended within a carrier material is generally directed to describing only the means by which it is physically, mechanically, or chemically bound within the carrier material. Further, it means that the second pharmacological agent is mechanically attached, entrapped, or otherwise tethered either to or within a nonliquid-phase region of the carrier material. Still further, when a pharmacological agent is suspend within a carrier material, the agent's dispersion can be either homogenous or heterogeneous.

The pharmacological agent can be, for example, a medicament, drug, or other suitable biologically, physiologically, or pharmaceutically-active substance, or any combination thereof, that is capable of providing local biological, physiological or therapeutic effect in the patient's body and of being released from the carrier material 12 into an adjacent target region of tissue. A non-exhaustive listing of example pharmacological agents that can be used as the first and second pharmacological agents include: an antineoplastic, anti-inflammatory, antiplatelet, anticoagulant, fibrinolytic, thrombin inhibitor, antimitotic, antiallergic, antistenotic, antibiotic, antiviral, analgesic, anesthetic, statin, antiproliferative substance, and any combination thereof. A particular pharmacological agent is CyA. The first pharmacological agent can optionally: be the same as the second pharmacological agent, be different than the second pharmacological agent, have a rate of release from the carrier material 12 that is the same as the rate of release of the second pharmacological agent from the carrier material 12, and have a rate of release from the carrier material 12 that is different than the rate of release of the second pharmacological agent from the carrier material 12, and any combination thereof.

According to another embodiment, the first pharmacological agent is generally homogeneously incorporated into the carrier material 12 and is different than the second pharmacological agent. The second pharmacological agent can be suspended within the carrier material 12 and contained within, attached to, form, or any combination thereof, microparticles, nanoparticles, gels, xerogels, bioadhesives and foams. All of the various forms the second pharmacological agent can be contained within, attached to, form, and any combination thereof, to be suspended within the carrier material 12 will be collectively referred to herein as a capsule 56. The capsule can be spherical, oblong, and any other desirable shape. Nonlimiting examples of the capsule include micro/nanoparticles, micro/nanospheres, micro/nanoencapsulating particles, micro/nanocapsules and liposomesa and are suspended within the carrier material 12 to be released from the carrier material 12 at a rate that differs from the release rate of the first pharmacological agent homogeneously incorporated therein. The term microsphere includes solid spheres formed of the second pharmacological agent, a polymer with the second pharmacological agent dispersed throughout, as well as microparticulates and microcapsules, as well as a particulate object having the second pharmacological agent provided on a surface. Specific examples of suitable materials for the formation of the capsule 56 include, but are not limited to, biodegradable polymers such as poly d,l-lactic acid (PLA) and copolymers of lactic acid and glycolic acid (PLGA).

A carrier material 12 provided with first and second pharmacological agents according to this embodiment will rapidly release the first pharmacological agent to the target region of tissue while providing a controlled, extended release of the second pharmacological agent as it escapes the encapsulation.

Although the capsule 56 mentioned above can be an enclosure formed from a suitable material around the second pharmacological agent, the capsule 56 of the present invention can optionally be a solid, gelatinous, and other form of particle formed from the second pharmacological agent itself, or in combination with any other suitable material mentioned herein. Further, the first and second pharmacological agents can be releasably incorporated within the carrier material with any type of suitable reversible linkage to the hydrogel-matrix structure to control the release of the first and second pharmacological agents from the carrier material 12. Examples of suitable reversible linkages include hydrogen bonds, ionic bonds, hydrolytically cleavable bonds, enzymatically cleavable bonds, physical entrapment within the hydrogel matrix which has been swollen by environmental factors such as temperature, pH, other similar techniques, and any combination thereof.

The release rate of the pharmacological agents from the carrier material 12 can be controlled, for example, by selecting a suitably soluble pharmacological agent for a particular hydrogel matrix, controlling the distribution of the pharmacological agents in the carrier material 12, encapsulating at least one of the pharmacological agents in a suitable capsule 56 and selecting a suitably sized, shaped, porous, soluble, biodegradable hydrogel matrix for the carrier material 12. Preferably, the device 10 will include a carrier material 12, a first pharmacological agent, and a second pharmacological agent that is encapsulated as a microsphere. When a first surface of the carrier material is placed in contact with the target region, the homogeneously incorporated first pharmacological agent will be rapidly released from the carrier material 12 relative to the release rate of the second pharmacological agent and introduced to the target region. As the first pharmacological agent is released, the second pharmacological agent begins to diffuse through the carrier material 12, and is released at the surface of the carrier material 12 in contact with the target region during a second period of time having a duration that is longer than the first period of time. According to this embodiment, a burst release of the first pharmacological agent during a first period of time is followed by the extended delivery of the second pharmacological agent during a desired second, extended period of time.

The first period of time can begin substantially immediately once contact between the first surface of the carrier material 12 and the target region of tissue has been established, and can last as long as desired. However, a preferable duration of the time period during which substantially all of the first pharmacological agent is released is less than about one month. Similarly, the extended release period of the second pharmacological agent from the carrier material 12 can begin at about the time contact between the first surface of the carrier material 12 and the target region is established, and can last as long as desired. But again, a preferable duration of the release period for substantially all of the second pharmacological agent to be released from the carrier material 12 is less than about 2 years. It is noted, however, that the start and finish times of the first and second periods of time during which the first and second pharmacological agents are released, respectively, can occur simultaneously, separately, and can overlap.

To minimize the release of the first pharmacological agent, the second pharmacological agent, or both, from the carrier material 12 at a second surface 62, which does not contact the target region when a first surface 66 of the carrier material 12 contacts the target region, a barrier layer can be provided to the second surface 62. As used herein, first surface 66 refers to the surface of the carrier material 12 that contacts the target region and through which at least one of the pharmacological agents is to be released. The second surface 62 can be any other surface of the carrier material 12 that is not in contact with the target region. Nonlimiting examples of how the barrier layer can be formed include coating the carrier material 12 by painting, spraying or subjecting the carrier material 12 to a dip-coating process; and forming a region in the hydrogel matrix that is impervious to the pharmacological agent(s) and prevents the pharmacological agent(s) from being released from the carrier layer 12 at the second surface. For the device 10 shown in FIG. 1, the barrier layer is provided on the outer periphery of the generally cylindrical carrier material 12. Arranged in this manner, the pharmacological agent(s) provided to the carrier material in FIG. 1 are prevented from being released from the carrier material 12 at the outer periphery. Suitable materials that can be used for the barrier layer include any that can minimize the release of a pharmacological agent at the second surface, such as polyurethane, polycarboxylic acid, polyorthoester, aliphatic polyester, polyanhydride, polysaccharide, polyamide, polyether, polyvinyl alcohol, polyethylene oxide, polyethylene glycol, protein, polypeptide, silicone based polymer, polyacrylamide, photopolymerizable monomer, polyglycolic acid, polylactic acid, poly(lactic-co-glycolic) acid, polycaprolactone, modified starch, gelatin, cellulose and its derivates, polyacrylic acid, polymethacrylic acid, polyhydroxybutyrate, polydioxanon, poly(ethylene vinyl acetate), polyethylene terephthalate, poly(vinylpyrrolidone), polytetrafluoroethylene, polyolefin, epoxide, poly(2-hydroxyethylmethacrylate) polyphosphazene polymer, fluoropolymer, polyamino acid, polyimine, polyphosphate, polysiloxane, polyvinyl ether, polyhydroxy acid, polyalkyl carbonate, albumin, fibrin, chitosan, alginate, poly(methylmethacrylate), collagen, polyphosphoester, hyaluronic acid, phospholipid, cyanoacrylate, polypropylene oxide. More preferably, however, the barrier layer includes polytetrafluoroethylene, polyurethane, polyamide, silicone based polymer, polyacrylamide, photopolymerizable monomer, polyethylene terephthalate, epoxide, fluoropolymer, polysiloxane, cyanoacrylate, and any combination thereof.

In addition to minimizing the release of a pharmacological agent at an undesired location, the material to be used for the barrier layer can be selected to protect the device 10 from immunological rejection by the body's immune system. In such a case, the barrier layer functions to prevent the immune system from recognizing the device 10 as a foreign object within the body, thereby shielding the device 10 from immunological attack. Examples of he types of preferred materials for protecting the device from the immune system in this manner include: polypropylene oxide, polyanhydride, polysaccharide, polyvinyl alcohol, polyethylene oxide, polyethylene glycol, polysiloxane, and alginate, however, other materials that can shield the device 10 from attacks by the immune system are also within the scope of the present invention.

From the above description of the invention, those skilled in the art will perceive improvements, changes and modifications. Such improvements, changes and modifications within the skill of the art are intended to be covered by the appended claims.

1. A medical device for delivering a pharmacological agent to a target region of tissue, the device comprising:
a resilient carrier material shaped to substantially encircle the target region;
a first pharmacological agent provided to the carrier material, wherein the first pharmacological agent is to be released from the carrier material during a first period of time and introduced to the target region.

2. The device according to claim 1 further comprising a second pharmacological agent dispersed within the carrier material to be released during a second period of time and introduced to the target region.

3. The device according to claim 2, wherein the second pharmacological agent is enclosed within a capsule that is suspended within the carrier material.

4. The device according to claim 3, wherein the capsule is a microsphere.

5. The device according to claim 2, wherein the first pharmacological agent is different than the second pharmacological agent.

6. The device according to claim 2, wherein the second period of time is longer than the first period of time.

7. The device according to claim 2, wherein the second period of time extends beyond the expiration of the first period of time.

8. The device according to claim 2, wherein the first period of time and the second period of time overlap.

9. The device according to claim 2, wherein the first period of time is less than about one month and the second period of time is less than about two years.

10. The device according to claim 1 further comprising a barrier layer to minimize the release of the first pharmacological agent from the carrier material at a surface not in contact with the target region.

11. The device according to claim 10, wherein the barrier layer comprises a material selected from the group consisting of polytetrafluoroethylene, polyurethane, polyamide, silicone based polymer, polyacrylamide, photopolymerizable monomer, polyethylene terephthalate, epoxide, fluoropolymer, polysiloxane, cyanoacrylate, polypropylene oxide, polyanhydride, polysaccharide, polyvinyl alcohol, polyethylene oxide, polyethylene glycol, polysiloxane, alginate, and any combination thereof.

12. The device according to claim 1, wherein the first pharmacological agent is selected from the group consisting of an antineoplastic, anti-inflammatory, antiplatelet, anticoagulant, fibrinolytic, thrombin inhibitor, antimitotic, antiallergic, antistenotic, antibiotic, antiviral, analgesic, anesthetic, statin, antiproliferative substance, and any combination thereof.

13. The device according to claim 1, wherein the first pharmacological agent is generally homogeneously incorporated throughout the carrier material.

14. The device according to claim 1, wherein the second pharmacological agent is selected from the group consisting of an antineoplastic, anti-inflammatory, antiplatelet, anticoagulant, fibrinolytic, thrombin inhibitor, antimitotic, antiallergic, antistenotic, antibiotic, antiviral, analgesic, anesthetic, statin, antiproliferative substance, and any combination thereof.

15. The device according to claim 1, wherein the carrier material is a hydrogel.

16. The device according to claim 15, wherein the hydrogel comprises an aqueous component and a polymeric component selected from the group consisting of a polyurethane, polycarboxylic acid, polyorthoester, aliphatic polyester, polyanhydride, polysaccharide, polyamide, polyether, polyvinyl alcohol, polyethylene oxide, polyethylene glycol, protein, polypeptide, silicone based polymer, polyacrylamide, photopolymerizable monomer, polyglycolic acid, polylactic acid, poly(lactic-co-glycolic) acid, polycaprolactone, modified starch, gelatin, cellulose and its derivates, polyacrylic acid, polymethacrylic acid, polyhydroxybutyrate, polydioxanon, poly(ethylene vinyl acetate), polyethylene terephthalate, poly(vinylpyrrolidone), polytetrafluoroethylene, polyolefin, epoxide, poly(2-hydroxyethylmethacrylate) polyphosphazene polymer, fluoropolymer, polyamino acid, polyimine, polyphosphate, polysiloxane, polyvinyl ether, polyhydroxy acid, polyalkyl carbonate, albumin, fibrin, chitosan, alginate, poly(methylmethacrylate), collagen, polyphosphoester, hyaluronic acid, phospholipid, cyanoacrylate, polypropylene oxide, and any combination thereof.

17. The device according to claim 1, wherein the carrier material comprises a first terminal-end portion positioned adjacent to a second terminal-end portion and defines a generally-cylindrical interior passage.

18. The device according to claim 1, wherein a longitudinal aperture is provided along the length of the carrier material to facilitate attachment of the device adjacent to the target region.

19. A device for delivering a pharmacological agent to a target region of tissue, the device comprising:
a pliant-material matrix to be positioned adjacent to the target region;
a first pharmacological agent generally homogeneously incorporated into the pliant-material matrix; and
a second pharmacological agent provided to the pliant-material matrix, wherein
the first pharmacological agent is released from the pliant-material matrix and introduced at the target region during a first period of time beginning substantially immediately after the pliant material is positioned proximate to the target region, wherein
the second pharmacological agent is released from the pliant-material matrix and introduced at the target region during a second period of time that extends beyond the expiration of the first period of time.

20. The device according to claim 19, wherein the second period of time begins after the first period begins.

21. The device according to claim 19, wherein the second period of time is longer than the first period of time.

22. The device according to claim 19, wherein the first period of time and the second period of time overlap.

23. The device according to claim 19, wherein the second pharmacological agent is provided as a microsphere suspended within the pliant-material matrix.

24. The device according to claim 19, wherein the first pharmacological agent is selected from the group consisting of an antineoplastic, anti-inflammatory, antiplatelet, anticoagulant, fibrinolytic, thrombin inhibitor, antimitotic, antiallergic, antistenotic, antibiotic, antiviral, analgesic, anesthetic, statin, antiproliferative substance, and any combination thereof.

25. The device according to claim 19 further comprising a barrier layer provided to a surface of the pliant-material matrix to minimize the release of at least one of the first pharmacological agent and the second pharmacological agent from the pliant-material matrix at an undesired location.

26. The device according to claim 19, wherein the pliant-material matrix defines a generally tubular interior passage in which at least a portion of the target region is enclosed.

27. The device according to claim 19, wherein the pliant-material matrix is a hydrogel matrix.

28. The device according to claim 27, wherein the hydrogel matrix comprises an aqueous component and a polymeric component selected from the group consisting of a polyurethane, polycarboxylic acid, polyorthoester, aliphatic polyester, polyanhydride, polysaccharide, polyamide, polyether, polyvinyl alcohol, polyethylene oxide, polyethylene glycol, protein, polypeptide, silicone based polymer, polyacrylamide, photopolymerizable monomer, polyglycolic acid, polylactic acid, poly(lactic-co-glycolic) acid, polycaprolactone, modified starch, gelatin, cellulose and its derivates, polyacrylic acid, polymethacrylic acid, polyhydroxybutyrate, polydioxanon, poly(ethylene vinyl acetate), polyethylene terephthalate, poly(vinylpyrrolidone), polytetrafluoroethylene, polyolefin, epoxide, poly(2-hydroxyethylmethacrylate) polyphosphazene polymer, fluoropolymer, polyamino acid, polyimine, polyphosphate, polysiloxane, polyvinyl ether, polyhydroxy acid, polyalkyl carbonate, albumin, fibrin, chitosan, alginate, poly(methylmethacrylate), collagen, polyphosphoester, hyaluronic acid, phospholipid, cyanoacrylate, polypropylene oxide, and any combination thereof.

29. A device for delivering a pharmacological agent to a target region of tissue, the device comprising:
a pliant material having a first surface to be placed in contact with the target region and a second surface that is not in contact with the target region when the first surface is in contact with the target region;
a first pharmacological agent provided to the pliant material, wherein the first pharmacological agent is to be released from the pliant material and introduced to the target region at an interface between the first surface and the target region; and
a barrier layer provided positioned to minimize release of the first pharmacological agent from the pliant material to an ambient environment at the second surface.

30. The device according to claim 29 further comprising a second pharmacological agent suspended within the pliant material.

31. The device according to claim 30, wherein the second pharmacological agent is suspended within the pliant material in a form selected from the group consisting of a microsphere, nanosphere, microencapsulating particle, nanoencapsulating particle, microcapsule, nanocapsule and liposome.

32. The device according to claim 30, wherein the second pharmacological agent is selected from the group consisting of an antineoplastic, anti-inflammatory, antiplatelet, anticoagulant, fibrinolytic, thrombin inhibitor, antimitotic, antiallergic, antistenotic, antibiotic, antiviral, analgesic, anesthetic, statin, antiproliferative substance, and any combination thereof.

33. The device according to claim 29, wherein the first surface is the periphery of a generally tubular interior passage adapted to encircle at least a portion of the target region.

34. The device according to claim 29, wherein the pliant material is a hydrogel matrix.

35. The device according to claim 34, wherein the hydrogel matrix comprises an aqueous component and a polymeric component selected from the group consisting of a polyurethane, polycarboxylic acid, polyorthoester, aliphatic polyester, polyanhydride, polysaccharide, polyamide, polyether, polyvinyl alcohol, polyethylene oxide, polyethylene glycol, protein, polypeptide, silicone based polymer, polyacrylamide, photopolymerizable monomer, polyglycolic acid, polylactic acid, poly(lactic-co-glycolic) acid, polycaprolactone, modified starch, gelatin, cellulose and its derivates, polyacrylic acid, polymethacrylic acid, polyhydroxybutyrate, polydioxanon, poly(ethylene vinyl acetate), polyethylene terephthalate, poly(vinylpyrrolidone), polytetrafluoroethylene, polyolefin, epoxide, poly(2-hydroxyethylmethacrylate) polyphosphazene polymer, fluoropolymer, polyamino acid, polyimine, polyphosphate, polysiloxane, polyvinyl ether, polyhydroxy acid, polyalkyl carbonate, albumin, fibrin, chitosan, alginate, poly(methylmethacrylate), collagen, polyphosphoester, hyaluronic acid, phospholipid, cyanoacrylate, polypropylene oxide, and any combination thereof.

36. The device according to claim 34, wherein the first pharmacological agent is chemically bonded within the hydrogel matrix, mechanically suspended within the hydrogel matrix, or both.

37. The device according to claim 36 further comprising a second pharmacological agent enclosed in a capsule that is mechanically suspended within the hydrogel matrix.

38. The device according to claim 29, wherein the first pharmacological agent is to be released during a first period of time substantially immediately after the first surface is placed proximate to the target region.

39. The device according to claim 29, wherein the first pharmacological agent is selected from the group consisting of an antineoplastic, anti-inflammatory, antiplatelet, anticoagulant, fibrinolytic, thrombin inhibitor, antimitotic, antiallergic, antistenotic, antibiotic, antiviral, analgesic, anesthetic, statin, antiproliferative substance, and any combination thereof.

40. A device for delivering a first pharmacological agent and a second pharmacological agent to a target region of tissue, the device comprising:
a carrier material having a first terminal-end portion positioned adjacent to a second terminal-end portion and defining a generally-cylindrical interior passage;
a first pharmacological agent provided to the carrier material, wherein
the first pharmacological agent is to be released from the carrier material during a first period of time and introduced to the target region;
a second pharmacological agent provided to the carrier material, wherein
the second pharmacological agent is released from the carrier material and introduced at the target region during a second period of time that extends beyond the expiration of the first period of time; and
a barrier layer provided to a surface of the carrier material to minimize release of at least one of the first pharmacological agent the second pharmacological agent from the carrier material to an ambient environment at an undesirable location.
